(19) United States
(12) Patent Application Publication
(10) Pub. No.: US 2006/0148089 A1
(43) Pub. Date: Jul. 6, 2006

(54) Small scale ideal kinetic reactor to evaluate full size industrial catalysts

(75) Inventors: Nallakkan Arvindan, San Francisco, CA (US); H. Sam Bergh, San Francisco, CA (US); Valery Sokolovskii, Sunnyvale, CA (US); Victor Wong, San Jose, CA (US)

Correspondence Address: SYMYX TECHNOLOGIES INC;LEGAL DEPARTMENT, 3100 CENTRAL EXPRESS, SANTA CLARA, CA, 95051, US

(73) Assignee: Symyx Technologies, Inc.

(21) Appl. No.: 11/322,039

(22) Filed: Dec. 28, 2005

Related U.S. Application Data
(60) Provisional application No. 60/640,428, filed on Dec. 30, 2004.

(51) Int. Cl. G01N 33/10

(52) U.S. Cl. 436/37

(57) ABSTRACT

The present invention is directed to an apparatus and method for obtaining kinetic information for commercial-form catalysts using minimal catalyst material while approximating large-scale reactor hydrodynamics and heat and mass transfer.

This application claims the benefit of U.S. Provisional Application No. 60/640,428, filed Dec. 30, 2004, which is incorporated herein by reference for all purposes.

### TECHNICAL FIELD

The present invention relates generally to systems for analysis of materials by contacting a plurality of solid members with a test fluid, and more particularly, to an apparatus and method for evaluating solid catalysts that have a size suitable for industrial scale processes in a laboratory scale reactor having hydrodynamics that perform comparably to a pilot or industrial scale fixed bed reactor.

### BACKGROUND OF INVENTION

In traditional catalyst development, researchers synthesize relatively small amounts of a candidate compound. They then test the compound to determine whether it warrants further study. For solid phase catalysts, this initial testing typically involves confining the compound in a crushed and sieved form in a vessel, such as a micro-reactor, and then contacting the compound with one or more fluid phase reactants at a particular temperature, pressure and flow rate. If the compound produces some minimal level of conversion to a desired product, the compound undergoes more thorough characterization in a later step.

The later step includes manufacturing the catalyst into shaped crystals, such as spheres, cylinders, or rings. These shaped catalysts are then evaluated in a bench top reactor for more thorough analysis. However, these reactions typically are not able to achieve ideal displacement conditions and/or are not conducted under real flow reaction conditions, such as those used in a pilot plant or industrial reactor. Typical reaction conditions for the bench top reactors include a lower fluid flow rate, a lower linear velocity, a diluted bed, and/or back-mixing.

It has been found that, in order for a fixed bed reactor, such as a tube, having a bed of catalyst grains, to have ideal displacement conditions, the diameter of the tube needs to be at least approximately 10 times greater, and the length of the bed needs to be at least approximately 30 times greater than the particle diameter. Thus, an industrial or pilot plant fixed bed reactor utilizing particles having a mass of at least 1 gram will require a large amount of catalyst material for reaction. This is not ideal for catalyst research, where amounts of material may be small, and a researcher needs to work in a laboratory, not a pilot plant.

M. I. Temkin, Laboratory Reactor With Ideal Displacement, Kinetika i Kataliz, vol. 10 no. 2, pp. 461-463 (1969), describes a laboratory-scale reactor in which a cylindrical reaction cavity is filled with industrial form catalyst beads that are spaced apart with inert cylinders. The reactor emulates a plug flow reactor by assembling a series of continuous stirred tank reactors. A large number of continuous stirred tank reactors in series behave as a plug flow reactor. However, under certain conditions, transport effects arising from poor mixing in stagnant zones in the reactor can impede accurate kinetic measurements.

Thus, there is a need for a reactor that can be used to obtain accurate kinetic information for industrial-form catalysts using minimal catalyst material while approximating large-scale reactor hydrodynamics as well as heat and mass transfer characteristics.

### SUMMARY OF INVENTION

It is therefore an object of the present invention to provide an apparatus and method for obtaining kinetic information for commercial-form catalysts using minimal catalyst material while approximating large-scale reactor hydrodynamics and heat and mass transfer.

Briefly, therefore, the present invention is directed to a chemical processing system comprising a plug flow reactor that comprises a surface defining a reaction cavity for carrying out a chemical reaction and containing industrial form catalyst particles, an inlet port in fluid communication with the reaction cavity, and an outlet port in fluid communication with the reaction cavity. The reaction cavity has a volume of not more than about 1 liter, and in some applications, not more than about 500 ml, 100 ml, 50 ml, 10 ml, 5 ml, 1 ml, 100 μl, 10 μl or 1 μl. A fluid distribution system can supply one or more reactants from one or more external reactant sources to the inlet port of the reaction cavity and can discharge a reactor effluent from the outlet port of the reaction cavity to one or more external effluent sinks.

The present invention is directed to single and parallel (e.g., multi-channel) chemical processing systems, especially, laboratory-scale chemical systems that approximate industrial scale process conditions. Specifically, the invention is directed to a laboratory-scale reactor system that approximates plug-flow behavior for industrial form catalysts while achieving space velocities and velocities over the surface of the catalyst particles comparable to those used in industrial processes. Although primarily discussed and exemplified herein in the context of laboratory-scale reactors, including parallel reactors, it is to be understood that the invention has applications in other chemical processing systems (e.g., mixing systems, separation systems, material-processing systems, etc.), some of which are discussed in varying detail below.

Briefly, therefore, in one embodiment, the present invention is directed to a method for evaluating catalysts, which includes flowing a reactant fluid through a reactor, wherein the reactant fluid contacts a plurality of solid catalyst particles located in a reaction cavity of the reactor under reaction conditions such that a flow characteristic of the reactant fluid through the reaction cavity of the reactor is a Peclet number for axial dispersion greater than 100 and in some aspects, greater than 125, 150, 175, 200, 225, 250, 275 or 300. The reactor includes a surface defining the reaction cavity, which has a volume less than 1 liter.

In another embodiment, the present invention is directed to a heterogeneous catalysis process, which includes contacting a gas stream in a reactor with a plurality of solid catalyst particles located in a reaction cavity of the reactor, the catalyst particles having dimensions suitable for industrial scale processes, under reaction conditions such that the gas stream has a substantially uniform velocity at all points over substantially the entire surface of each of the plurality of solid catalyst particles. The reactor includes a surface defining the reaction cavity, which has a volume less than 1 liter.

In another embodiment, the present invention is directed to a method for evaluating catalysts, which includes flowing a reactant fluid through a reactor that comprises a surface defining an internal cavity, and an insert located in the internal cavity. The insert includes a fluid inlet for receiving a fluid from a fluid source, a fluid outlet for discharging the fluid as an effluent, a surface defining a plurality of reaction cells connected via conduits, and a plurality of industrial form solid catalyst particles located in the reaction cells. Each reaction cell is adapted to hold a single catalyst particle. The reactant fluid flows through the reaction cells, and contacts the plurality of solid catalyst particles located in the reaction cells under reaction conditions.

In another embodiment, the present invention is directed to a system for contacting solid catalyst particles with a fluid. The system includes a reactor adapted to hold a plurality of solid catalyst particles, and includes a surface defining an internal cavity, and an insert located in the internal cavity. The insert includes a fluid inlet for receiving a fluid, a fluid outlet for discharging the fluid, and a surface defining a plurality of reaction cells connected via conduits. Each reaction cell is adapted to hold a single catalyst particle.

In another embodiment, the present invention is directed to a reactor system, which includes a vessel comprising an inlet for receiving a fluid, an outlet for discharging the fluid as an effluent, a surface defining a reaction cavity having a volume less than 1 liter, and a plurality of industrial form catalyst particles located in the reaction cavity. The reaction cavity is adapted to provide a substantially uniform velocity of the fluid over the surface of each of the catalyst particles under reaction conditions.

In another embodiment, the present invention is directed to a reactor system for evaluating catalysts. The reactor includes a vessel comprising an inlet for receiving a fluid, an outlet for discharging the fluid, a surface defining a reaction cavity, and a plurality of solid catalyst particles all having approximately the same geometry and dimensions located in the reaction cavity. The reaction cavity has a cross sectional area no greater than twice a cross-sectional area of a single catalyst particle of the plurality of catalysts, and a length adapted to accommodate the plurality of catalyst particles. The reaction cavity is adapted to provide a substantially uniform velocity of the fluid over substantially the entire surface of each of the plurality of catalyst particles under reaction conditions.

In another embodiment, the present invention is directed to a parallel flow reactor system for evaluating a plurality of industrial form catalyst particles. The reactor has a plurality of surfaces defining a plurality of reaction cavities, each of the plurality of reaction cavities having an inlet for receiving a reactant-containing stream and an outlet for discharging a product-containing stream. Each reaction cavity has a plurality of industrial form solid catalysts contained therein. The reactor system is adapted such that each reactant-containing stream can be fed through the plurality of reaction cavities simultaneously to contact the catalyst particles under reaction conditions and each reaction cavity is adapted to provide a substantially uniform velocity of the reactant-containing stream over substantially the entire surface of each of the plurality of catalyst particles under reaction conditions.

In some embodiments, by designing the geometry of a reaction cavity in a small scale reactor to accommodate industrial form solid catalyst particles and by controlling the hydrodynamics of the reaction to model an ideal plug flow reactor, relevant performance characteristics and accurate kinetic data can be obtained using a small-scale reactor. Thus, catalysts that would typically be run in a pilot plant can be evaluated on a much smaller scale, and the data obtained can be useful in predicting the performance of the same catalyst particles in larger reactors.

In some embodiments, the reaction cavity has a shape that that roughly follows the contour of the catalyst particles such that a fluid velocity and residence time behaves in a plug flow manner. Some embodiments utilize a cavity chain reactor, which is a plurality of reaction cavities or reaction cells, arranged in a chain formation. Each cavity or cell in the chain is designed to hold a catalyst particle and has a shape that that roughly follows the contour of the catalyst particle.

One advantage of the present invention is that the hydrodynamics perform comparably to a pilot or industrial scale fixed bed reactor. The velocity of the reaction fluid over the surface of the catalyst is similar to a pilot or industrial scale reactor at a given space velocity such that the heat and mass transfer characteristics are attained on a laboratory-scale. With this reactor an average linear velocity greater than 1 meter/second can be obtained at a space velocity at 5000 per hour.

Another advantage of the present invention is a very fast experiment turnaround. Catalysts can be changed and the reactor can be cleaned and/or re-started typically in less than 30 minutes.

The present invention also provides the ability to monitor individual catalyst temperatures in-situ, thus yielding additional kinetic and transport phenomena information.

The present invention provides a bench top reactor providing more uniform mass transfer over the entire surface of the catalyst particles than do other bench top reactors for evaluating industrial form catalysts.

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A through FIG. 1D are cross-sectional side views showing exemplary embodiments of reactor systems comprising solid catalyst particles.

FIG. 2A is a perspective views of an embodiment of a body of a reactor of the present invention with various elements shown in phantom.

FIG. 3A through FIG. 3C are elements of an insert for use in reactors of the present invention. FIG. 3A is a perspective view of two halves of an insert used to hold spherical catalyst particles and define the reaction cavity in reactors of the present invention. FIG. 3B is a blown up view if two reaction cells of the insert shown in FIG. 3A. FIG. 3C is a perspective view of a sleeve that holds the insert.

FIG. 4A through 4F are perspective views of various end caps for use with reactors of the present invention. FIG. 4A is a general view of end caps for use in the present invention. FIG. 4B is a general view of end caps for use in the present invention showing various assembly details. FIG. 4C is a general view of end caps for use in the present invention showing the general fluid paths through the end cap. FIG. 4D is a view of an embodiment of an end cap for use in the present invention for connection to a fluid source, and for discharging an effluent to an analyzer. FIG. 4E is a view of an embodiment of an end cap for use in the present invention utilizing a connecting path for connecting two reaction cavities in a block. FIG. 4F is a view of an embodiment of an end cap for use in the present invention for connecting two reaction cavities in two separate blocks.

FIG. 5 is a perspective view of a sealing plate for use in some embodiments of reactors of the present invention.

FIG. 6A and FIG. 6B are views of an embodiment of the present invention combining two blocks using various end caps to create a single, continuous reaction cavity out of four reaction cavities. FIG. 6A is a side view of the reactor. FIG. 6B is a perspective view of the reactor showing the reaction path in phantom.

FIG. 7 is a graph showing residence time distribution data for the experiment run in Example 1.

FIG. 8A through FIG. 8E are graphical data for example 2 run on the reactor of the invention. FIG. 8A and FIG. 8B show the mole fractions of nitrogen at the inlet and outlet of the reactor of the invention as a function of time when the gas is switched to nitrogen. FIG. 8C shows calculated RTD for the reactor of the invention having up to 140 beads, or catalyst particles. FIG. 8D shows the simulated axial velocity profile between a single bead and the reactor wall in the reactor of the invention, and FIG. 8E shows a blown up view of the profile shown in FIG. 8D.

FIG. 9A is a schematic embodiment of a reactor known in the prior art and used as a comparison to the reactor of the invention in example 2. FIG. 9B shows calculated RTD for the reactor of the prior art having up to 140 beads, or catalyst particles. FIG. 9C shows the simulated axial velocity profile between a bead and the reactor wall in the prior art reactor, and FIG. 9D shows a blown up view of the profile shown in FIG. 9C.

FIG. 10A shows the comparison of calculate Peclet number for the reactor of the invention and the prior art reactor as a function of number of beads under a similar flow rate of 1 liter/minute. FIG. 10B shows the comparison of calculated Peclet number for the reactor of the invention and the prior art reactor as a function of number of beads using a similar residence time for each reactor.

### DETAILED DESCRIPTION OF THE INVENTION

The present invention provides an apparatus and method for reacting a plurality of industrial form catalysts in a laboratory scale reactor while closely approximating industrial or pilot plant reactor conditions. This is accomplished by controlling the hydrodynamics to model an ideal plug flow reactor. “Plug flow” characterizes the manner in which materials move through a reactor. Due to the configuration and/or operation of the reactor, any particular selected plug of material traversing the reactor has minimal axial mixing with an adjacent plug of material even though there may be radial mixing within the plug.

One way of characterizing flow characteristics of a fluid through a reactor is by the Peclet number for axial dispersion. The Peclet number for axial dispersion (a dimensionless number) is a ratio comparing the relative effects of convective fluid flow versus axial dispersion. When the ratio is low, the Peclet number for axial dispersion is low, and dispersion is more pronounced. Dispersion results in a spread of the residence time that different slices, or plugs, of the fluid spend in the reactor. In the context of chemical reactions, higher dispersion can result in lower conversions. Hence, the lower the Peclet number for axial dispersion, the lower the maximum conversion that can be attained in the reactor.

Chemical reactors are classified by the type of mixing as plug flow reactors (little or no intermixing of fluid) and continuous stirred tank reactors (CSTR) (complete mixing). An ideal plug flow reactor has a very high Peclet number for axial dispersion, typically greater than 100. Because pilot plant and large-scale packed beds exhibit plug-flow behavior with very few exceptions, plug flow is desired in a laboratory scale reactor. Thus, the Peclet number for axial dispersion is used to characterize how close a reactor is to a plug flow reactor for the reactions of interest to the present invention.

Another characteristic of importance in reactors of the present invention is having a substantially uniform velocity of the fluid at all points over the surface of each catalyst particle at a given space velocity. Velocity of the fluid at a given point is defined as the volumetric flow rate of the fluid divided by the cross-sectional area of the fluid path at that point. Space velocity is defined as the volumetric flow rate of the fluid divided by the volume of the catalyst particle. Reactors of the present invention are able to run at space velocities comparable to space velocities run in industrial reactors, while maintaining a substantially uniform velocity of the fluid at all points over the surface of each catalyst particle comparable to those attained in industrial reactors.

In one aspect, the invention simulates the hydrodynamics of large scale reactors using industrial-form catalyst particles in a laboratory-scale reactor. In one embodiment, this is accomplished by using a modular ball cavity chain reactor. The reactor can test industrial-form catalysts (beads, cylinders, extrudates, etc.) without diluent while maintaining plug-flow characteristics under reaction conditions.

By “industrial form catalyst particles” it is intended to mean catalyst particles having dimensions suitable for industrial scale processes. This includes catalyst particles having a mass of at least 1 g, and/or having a diameter of at least 3 mm. In preferred embodiments, catalyst particles are spherical and have a diameter between about 3 mm and 10 mm, and more preferably between about 3 mm and 8 mm.

In one embodiment, reaction is achieved by providing a laboratory-scale or bench top reactor having a reaction space geometry designed to have a shape that roughly follows the contours of the catalyst particles such that the fluid residence time behaves in a plug flow manner. Reactions can be simultaneous for two or more pluralities of catalysts located in a plurality of reaction cavities, or carried out in a rapid serial manner. Changes in the fluid resulting from contact with the catalysts are used to evaluate the catalysts. In the following disclosure, the term “fluid” refers to any substance that will deform continuously under the action of a shear force, including both gases and liquids.

The apparatus and method can be used to evaluate catalysts based on any property that can be discerned by detecting or measuring changes in a test fluid following contact with a plurality of catalyst particles. Thus, for example, the catalyst particles can be evaluated for catalytic activity by contacting the catalyst particles with a reactive fluid and detecting conversion or selectivity.

The disclosed invention is not limited to evaluating catalysts, but can be used for reaction of many different types of materials. For example, the method and apparatus can be used to evaluate solid particles based on their ability to filter out or adsorb a specific gas species. The concentration of that gas species in a fluid stream following contact with a plurality of solid particles is inversely proportional to the particular material's performance. Similarly, for example, polymeric materials can be evaluated for thermal stability by measuring the concentration of gaseous decomposition products in an inert fluid stream in contact with heated library members. The amount of decomposition product evolved by a particular polymeric material is a measure of that material's thermal stability.

The invention is described in further detail below with reference to the Figures, in which like items are numbered the same in the several figures.

Reactor Configurations

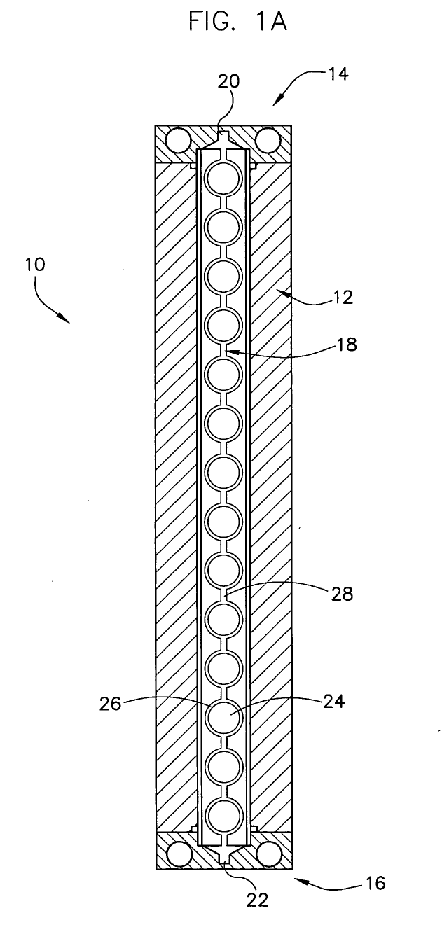

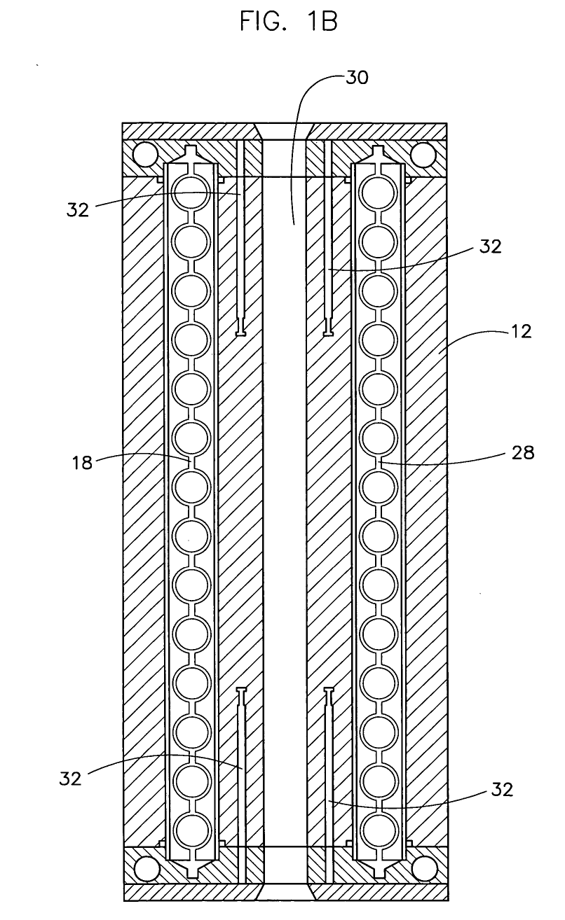

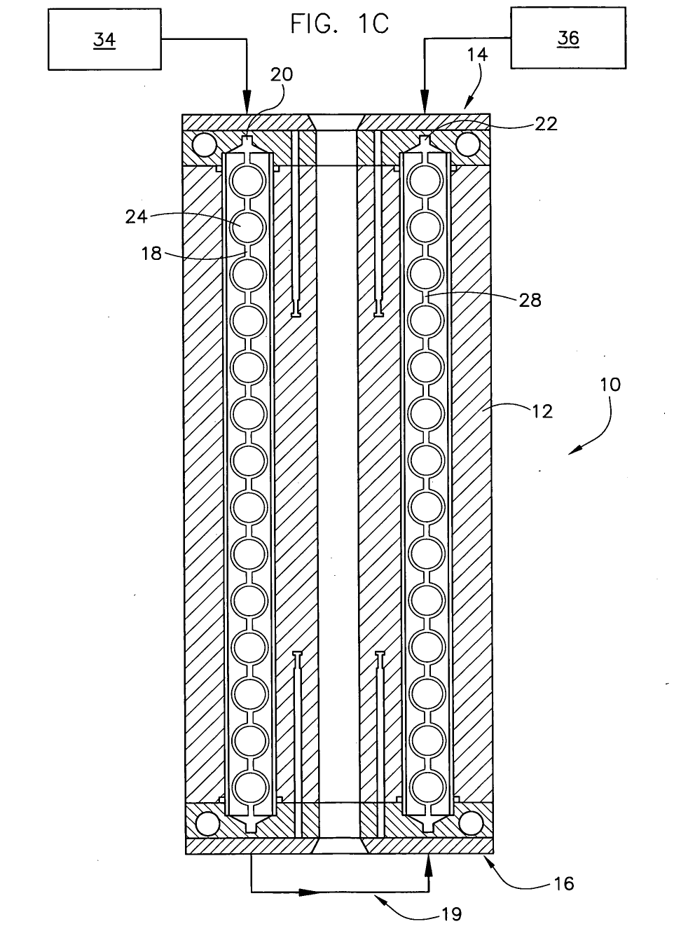

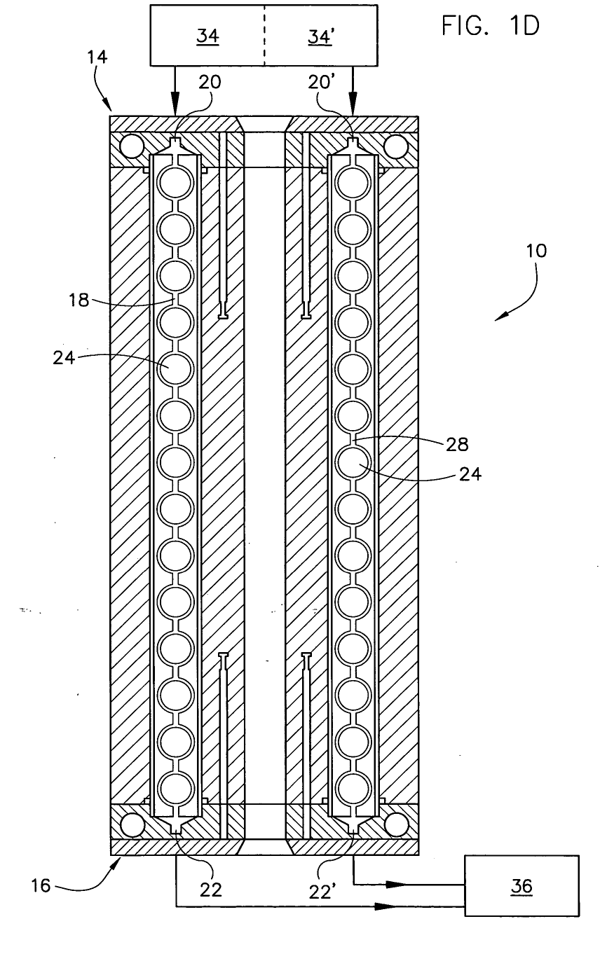

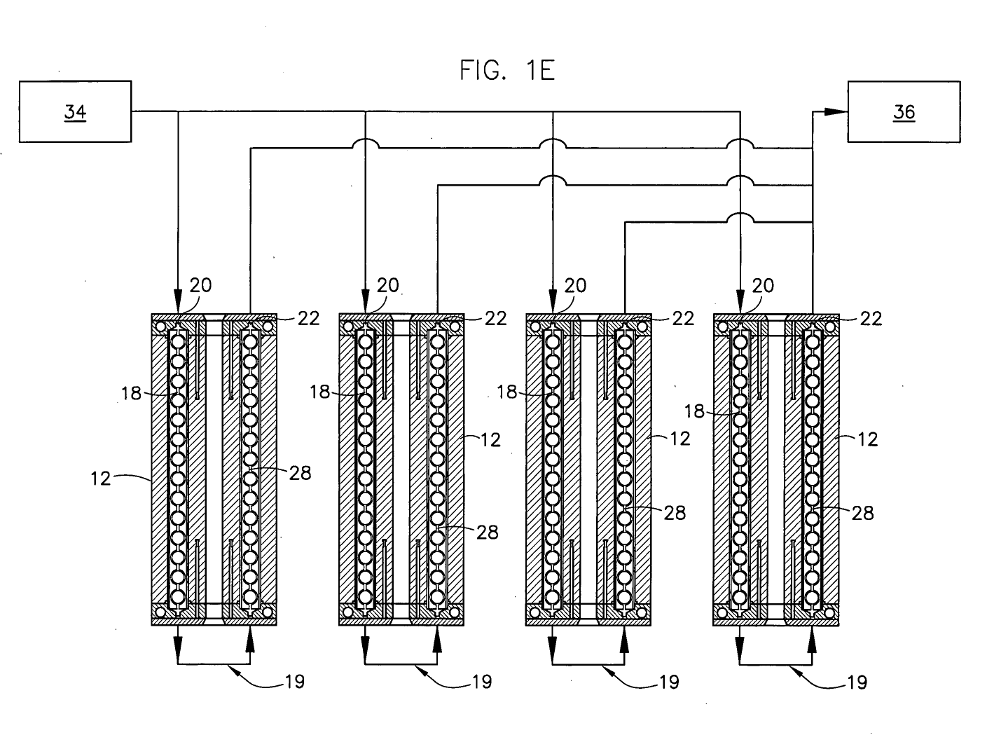

FIG. 1A through FIG. 1D are cross-sectional side views showing exemplary embodiments of reactor systems of the present invention. FIG. 1A shows an embodiment of an apparatus for evaluating a plurality of industrial form catalyst particles for properties such as activity or selectivity. The reactor 10 shown in FIG. 1A having a first end 14, and a second end 16 includes a body 12, such as a block, or module. The body 12 has a surface defining a reaction cavity 18 extending through the body 12. The reactor has an inlet 20 in fluid communication with the reaction cavity 18 adapted to receive fluid from a fluid source, and an outlet 22, in fluid communication with the reaction cavity 18 and adapted to discharge effluent from the reaction cavity 18. The reaction cavity 18 is designed to hold a plurality of catalyst particles 24.

The reaction cavity 18 shape can be designed based on the geometry of the solid catalyst particles 24 contained therein. For example, the embodiment shown in FIG. 1A has a reaction cavity designed to evaluate spherical catalyst particles 24 as shown by the shape of the reaction cavity 18. In the embodiment of FIG. 1A, the reaction cavity 18 is made up of a plurality of regions (i.e., reaction cells or reaction chambers) 26, each region 26 designed to hold a single solid (e.g., catalyst) particle 24, with a conduit 28 connecting each region 26. The regions 26 are designed to roughly follow the shape of the catalyst particle 24 so that under reaction conditions, fluid flow will have a substantially uniform linear velocity at over substantially the entire surface of each catalyst particle. This design also minimizes stagnant or dead zones in the reaction cavity 18. Thus, the embodiment shown in FIG. 1A, designed to evaluate spherical catalyst particles 24, has spherical regions 26. Other catalyst particle geometries are known in the art and are intended to be within the scope of the present invention.

In other embodiments, as shown in FIGS. 1B through 1E, the reactor body 12 has a surface defining a second reaction cavity 28. In addition, the body, whether a single cavity reactor or a reactor with a plurality of reaction cavities, may also optionally have a surface defining a cavity for containing a heater cartridge 30 so that the reactor may be run at high temperatures in the absence of an oven, as well as a plurality of surfaces defining cavities for containing thermocouples 32 for monitoring the temperature of the reactor at various locations.

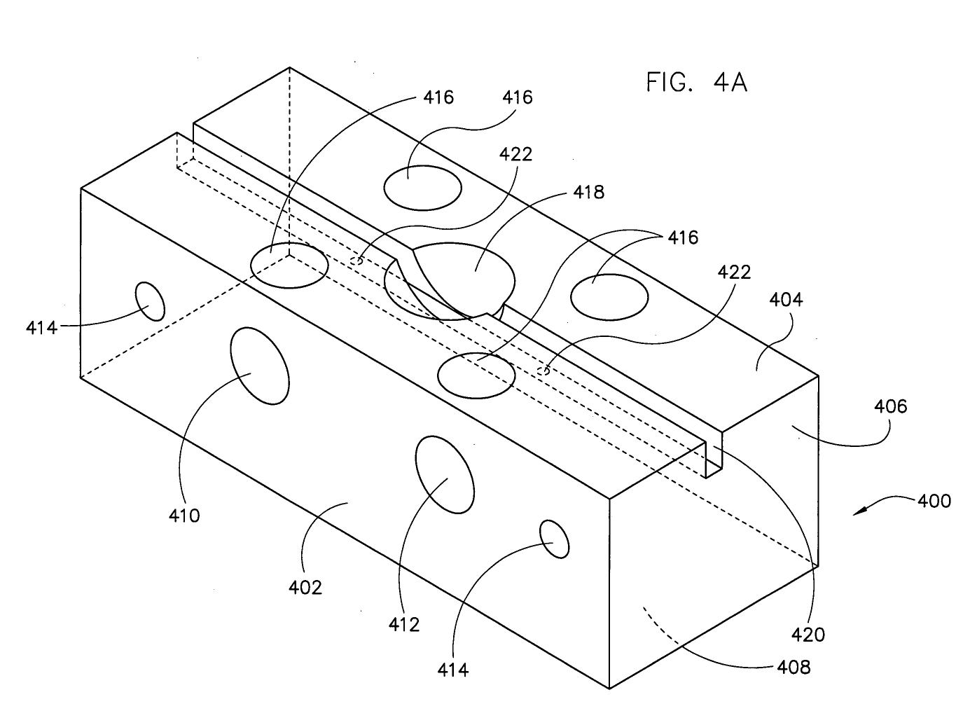

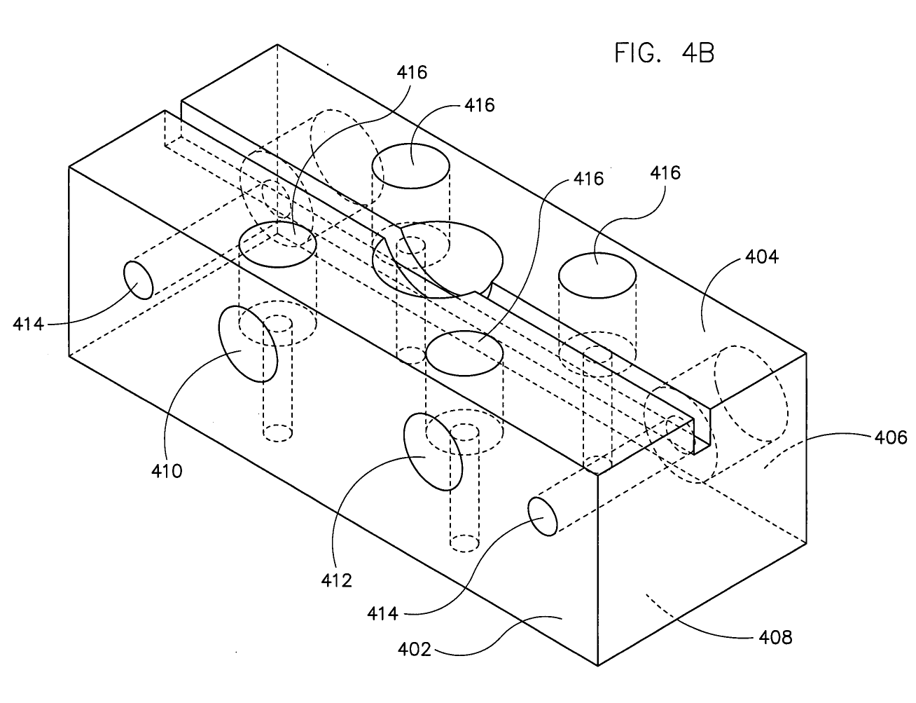

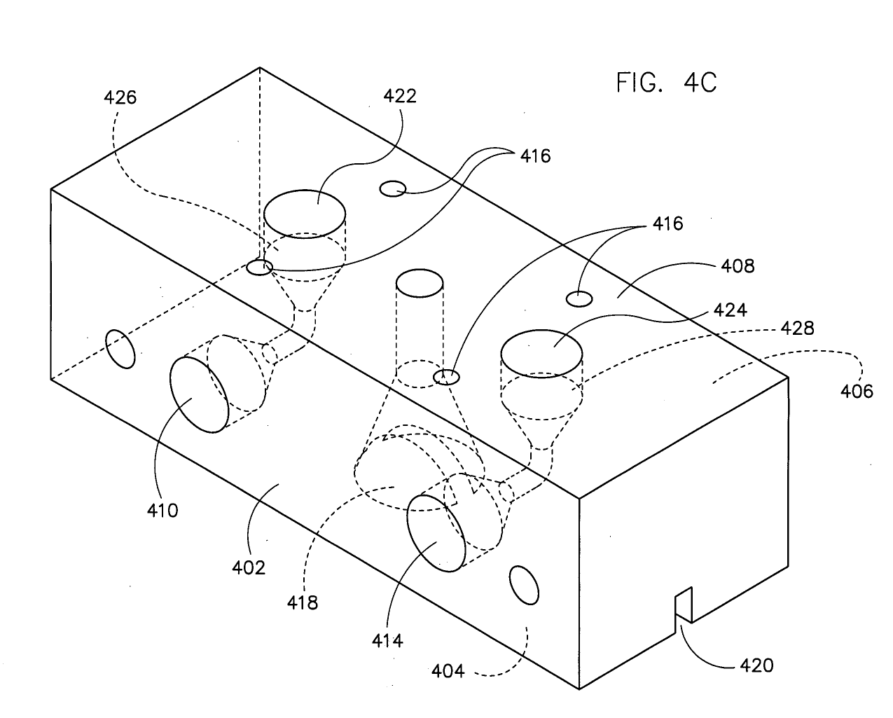

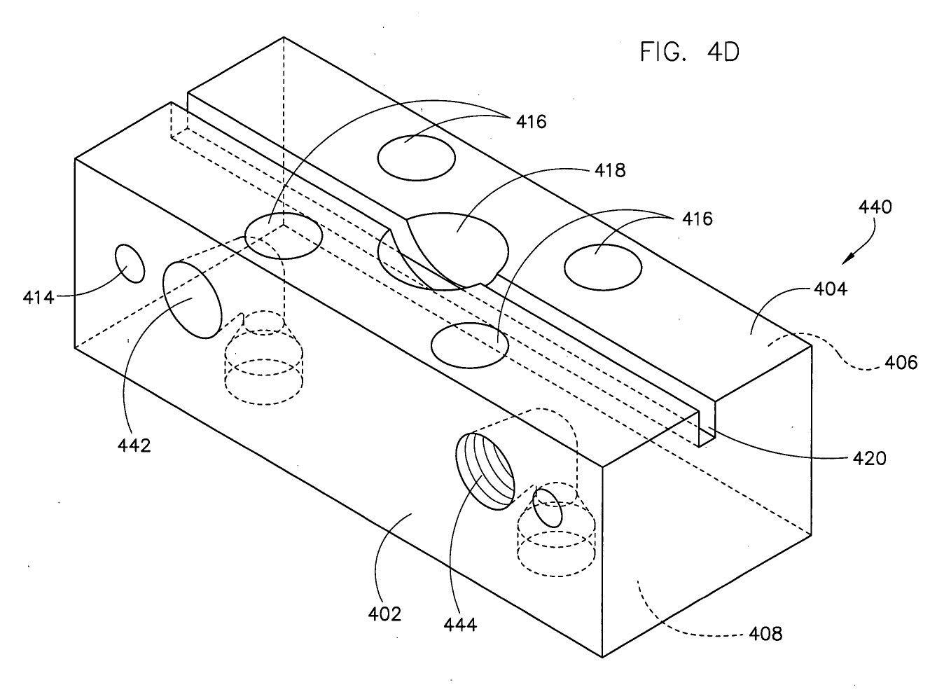

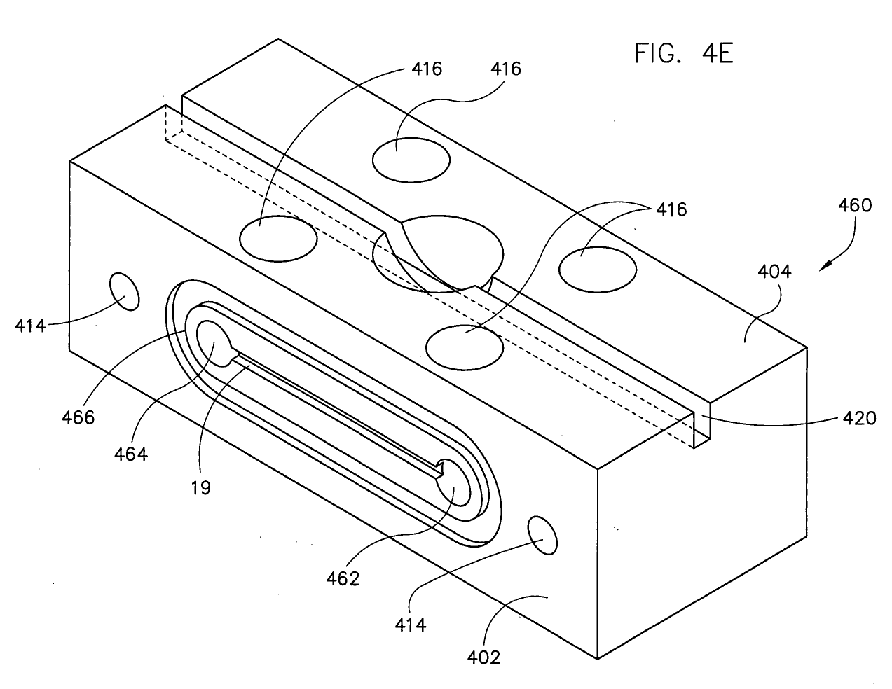

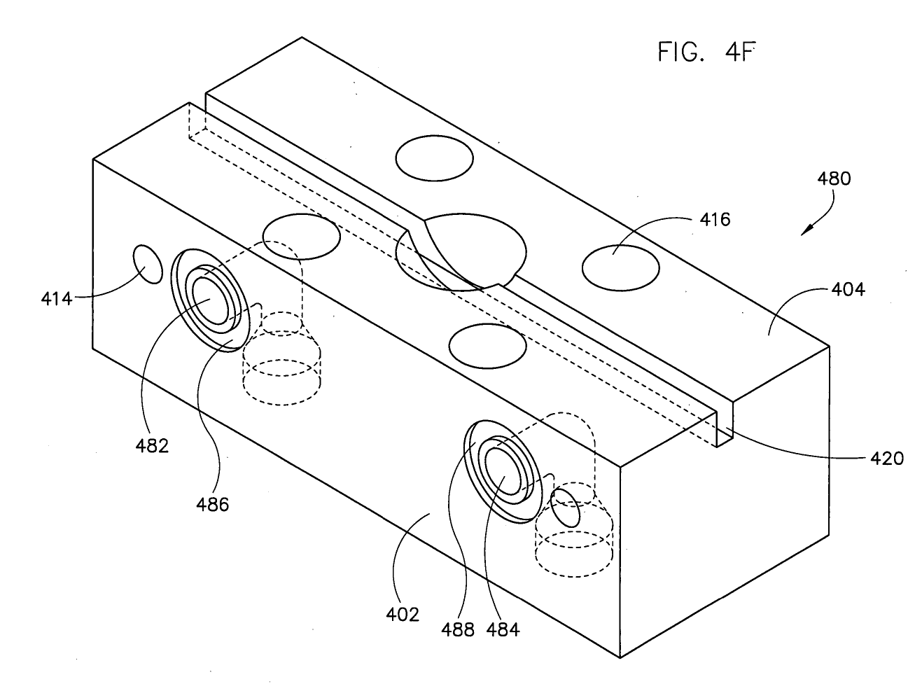

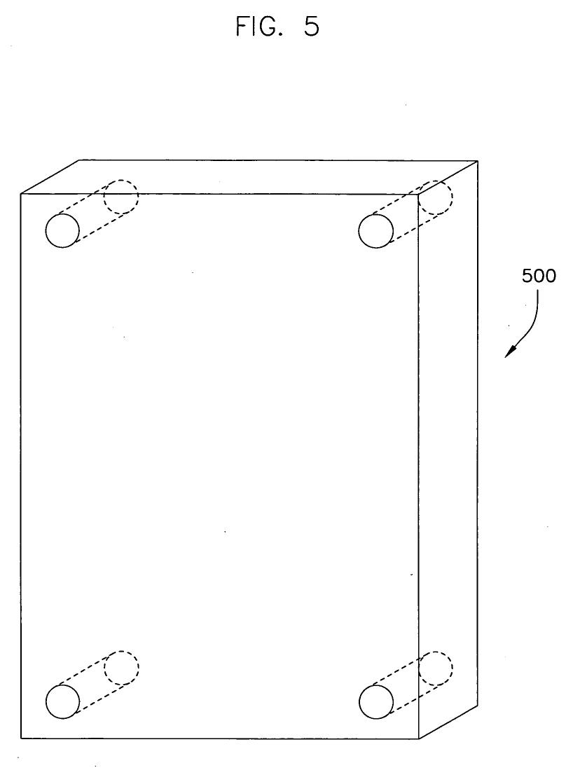

The reactor 10 of the invention can be utilized in several different ways. For example, in one embodiment, as shown in FIG. 1C, the reaction cavities 18 28 can be combined to be a single reaction cavity for a single reaction. In this embodiment, the inlet 20 and the outlet 22 are both located on the first end 14 of the reactor 10. The reaction cavities 18 28 are connected by a connecting cavity 19 (shown only schematically here) located at the second end 16 of the reactor 10. Fluid, such as a reactant gas flows from the fluid source 34 through the inlet 20 and through the reaction cavities 18 19 28 where it is brought into contact with the plurality of solid particles 24, such as catalyst particles. The fluid flows from the outlet 22 as an effluent, preferably to a detector 36, such as a gas chromatograph. The specific inlet, outlet and connecting cavity 19 configurations are described in detail with reference to the description of the end caps in FIG. 4A through FIG. 4F.

In another embodiment, as shown in FIG. 1D, the reaction cavities 18 28 can be two separate reaction cavities for two separate reactions. In this embodiment, the reactor 10 comprises two inlets 20 20′ located at the first end 14 of the reactor 10, and two outlets 22 22′ located at the second end 16 of the reactor 10. For running 2 reactions, the two inlets 20 20′ may both be connected to a common fluid source, or may alternatively be connected to separate fluid sources that provide similar or different feeds. Fluid, such as a reactant gas flows from the fluid source(s) 34 34′ through the inlets 20 20′ and through the reaction cavities 18 28 where it is brought into contact with a plurality of solid particles 24 24′, such as catalyst particles. The fluid flows from the outlets 22 22′ as an effluent, preferably to one or more detectors 36, such as a gas chromatograph, where the effluent streams can be analyzed in a serial or parallel fashion.

The reactor 10 of this embodiment can be utilized in several ways. For example, the reactor 10 can be utilized to run two reactions simultaneously in a parallel fashion, by running two separate feeds, either from separate fluid sources or from a common fluid source through the reaction cavities 18 28, and running the effluent streams to a parallel or rapid serial detector. Alternatively, the reactor 10 can be utilized to run two reactions in a serial fashion, running the reactant gas through the first reaction cavity 18, then switching to the second reaction cavity 28 after the first reaction is through.

In another embodiment, as shown in FIG. 1E, a plurality of bodies 12 can be combined to run several reactions simultaneously. This embodiment includes a plurality of reactors 10, each reactor having a configuration as described in FIG. 1C. This embodiment is useful for running parallel reactions while gaining a longer residence time than the embodiment shown in FIG. 1D. In this embodiment, a plurality of reactant feeds, whether the feeds are from a common fluid source or from separate sources, are fed to the inlets of each reactor 10 and simultaneously contact the catalysts located in each reaction cavity 18. The effluent from each cavity runs to one or more analyzer 36, where the effluents are analyzed in parallel or in serial fashion.

While the embodiment shown and described in FIG. 1E includes a plurality of bodies 12, it is also possible for everything to be contained in a single body 12 having, for example, eight reaction cavities.

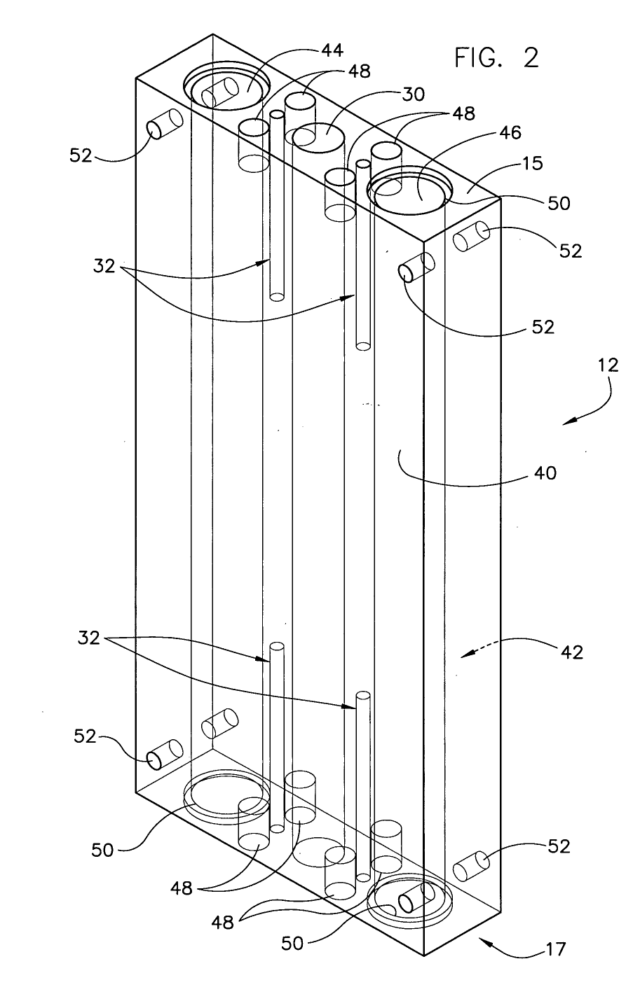

FIG. 2 through FIG. 5 show various embodiments of reactor components that can be utilized to attain the different configurations discussed above.

Body

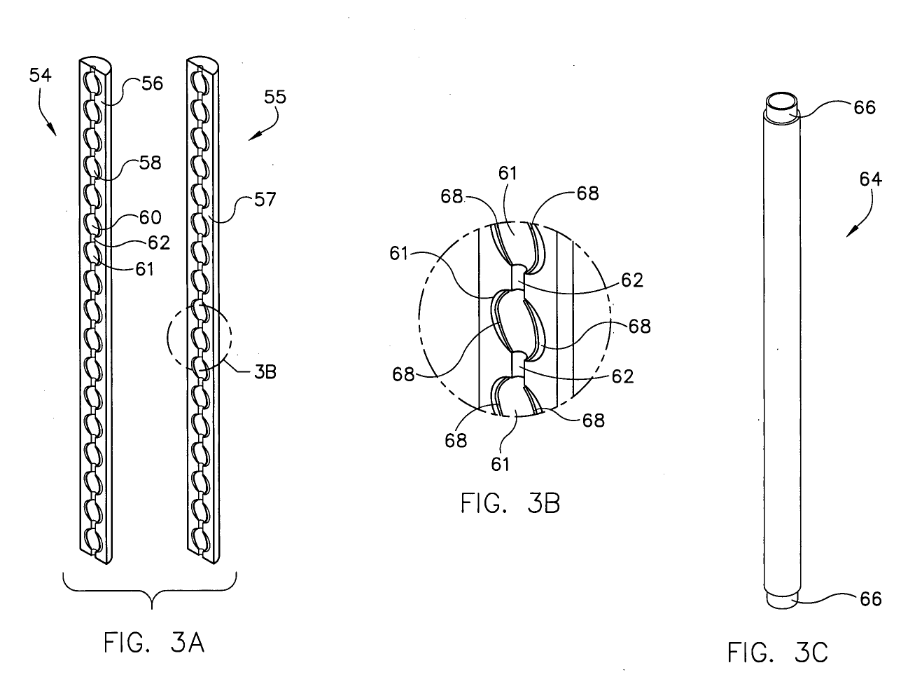

FIG. 2 shows an embodiment of a body 12 of the reactor 10 of the present invention having two reaction cavities. The body 12 in this embodiment is a block, although other geometries are well within the scope of the present invention. The body 12 may be constructed of materials having a relatively high thermal conductivity, for example, to provide for efficient heat transfer and a large thermal mass. Copper, stainless steel, aluminum or other metals are exemplary suitable materials for this embodiment, and may be coated with one or more other materials (e.g. nickel-coated copper) to provide additionally desired properties (e.g., chemical inertness) in combination. Materials that are at least machinable (on a macro-scale) are likewise preferred, to provide for assembly and other features. The block has a first end 15, a second end 17 parallel to and opposite the first end 15, a first face 40, which is perpendicular to the first and second ends, and a second face 42, which is parallel to and opposite the first face 40. The block has two throughbores 44 46 running from the first end 15 to the second end 17 and in a plane parallel to the first face 40 and second face 42. The throughbores 44 46 can define the reaction cavities 18 28 as described in FIG. 1B, or they can be designed to hold an insert which holds the catalyst particles and defines the reaction cavity. The inserts are described in detail below with reference to FIG. 3A through FIG. 3C. The block also optionally has a surface defining a cavity for containing a heater cartridge 30 so that the reactor may be run at high temperatures in the absence of an oven, as well as a plurality of surfaces defining cavities for containing thermocouples 32 for monitoring the temperature of the reactor at various locations of the body 12. For assembly, the block also may have a means of connecting the block to end caps. In the embodiment shown, the block has four threaded cavities 48 for receiving set screws on each end 15 17. The threaded cavities 48 complement set screw cavities in the end caps (described in detail below) and are designed to receive set screws for maintaining a connection with the end caps. Other connection means are known to those of skill in the art and are within the scope of the invention. In order to maintain pressure and a fluid seal in the reaction cavities 18 28 when in connection with the end caps, the throughbores 44 46 are designed to have O-ring grooves 50 at the first and second ends 15 17. An o-ring is placed in the groove and maintains a fluid and pressurized seal with the end caps. The first and second faces 40 42 can optionally have alignment means 52 for aligning two block together or for aligning a plate to the face. In the embodiment shown, the alignment means 52 is a small cavity designed to hold a pin and set in alignment with a complementary cavity on a second block or plate. Small pins can be placed inside the complementing cavities and can keep the blocks in alignment. Other ways of aligning the blocks are known, and are within the scope of the invention, such as complementing male and female connectors.

Insert

FIG. 3A through FIG. 3C show elements of one embodiment of an insert for use in reactors of the present invention. In one embodiment, the insert is made up of two halves 54 55 that are combined to hold the catalyst particles and define the reaction cavity 18. FIG. 3A shows a first insert half 54 and a second insert half 55, designed to hold spherical catalyst particles and combine to create the insert. The first insert half 54 has a face 56 which has a surface defining a recess 58 which defines half of the reaction cavity. The recess 58 is made up of a plurality of half spheres 60 connected by channels 62. The face 56 of the first insert half 54 is combined with an identical second insert half 55 of an insert by bringing the face 56 of the first insert half into contact with a corresponding face 57 of the second insert half. When brought into contact, the recesses in the faces form the reaction cavity 18 in a cavity chain configuration. The reaction cavity 18 is made up of a plurality of reaction cells connected to each other via conduits. In this embodiment, each reaction cell is designed to hold one catalyst particle. It is advantageous to use two halves for the insert so that catalyst particles may be set into and/or removed from the reaction cavity easily. In practice, catalyst particles are set into the reaction cells of the first half 54 of the insert. Catalyst particles may be set in every half sphere 60, or alternatively some half spheres may contain inert spacers, such as glass beads, or may remain empty. Once the catalyst particles are set in the first insert half 54, the second insert half 55 is brought into contact with the first insert half 54, thus creating an insert having a reaction cavity containing the catalyst particles.

The resulting insert may then be slid into a sleeve 64 as shown in FIG. 3C. The sleeve 64 is designed to hold the first and second insert pieces tightly together and also to fit in the throughbore 44 46 of the body 12. The sleeve 64 can have an O-ring groove 66 on each end in order to complement the O-ring grooves 50 in one of the throughbores 44 46 of the body 12.

Like the body 12, the insert and sleeve 64 are preferably constructed of materials that are machinable, have good thermal conductivity, and are relatively chemically inert, with aluminum being preferred.

The reaction cavity 18 of the reactor is designed so as to hold the catalyst particles and have a reaction pathway around each catalyst particle so that under reaction conditions, a Peclet number for axial dispersion greater than 100, specifically more than 125, more than 150, more than 175, more than 200, more than 250, and more than 300 is achieved. Space velocities such as those run in industrial reactors are achieved, and at those space velocities, there is a substantially uniform velocity at all points over the surface of each catalyst particle. The insert design of FIG. 3A accomplishes these objectives as is shown below in the examples. The walls of the reaction cells 61 are contoured to roughly follow the shape of the catalyst particles contained therein. It is desirable to hold the catalyst particles in the reaction cavity and maintain a substantially uniform distance between the cavity wall and the catalyst surface. This can be accomplished using spacers as shown in FIG. 3B, which is a blown up view of a portion of the insert half 55. In one embodiment, wire spacers 68 are used to hold the spherical catalyst particles in a set position in each reaction cell 61 of the reaction cavity. Two spacers 68 are 45 degrees offset from the face 56 of the insert half in the half sphere that makes up half of a reaction cell. The wire spacers 68 are suitably attached to the insert half, such as by soldering. In order to promote uniform fluid flow through the reaction cavity, the ends 70 of the spacers 68 are preferably flush with the face of the conduit. When the two insert halves 54 55 are combined, each reaction cell has 4 wire spacers 68 for holding the catalyst particles.

The design of the insert as described above, in combination with the use of spherical catalyst particles sized appropriately to the reaction cavity size, allows for ideal plug flow conditions during reaction and a substantially uniform velocity at all points over the surface of each catalyst particle at space velocities run in industrial and pilot plant reactors. In a preferred embodiment, the cross sectional area of the reaction cavity at any point is less than three times, preferably less than twice, the cross-sectional area of the largest catalyst particle located in the reaction cavity. In preferred embodiments, the catalysts are industrial form catalysts all having approximately the same dimensions, geometry, and chemical compositions. It may be desirable however, to react catalysts having different geometries, dimensions and/or chemical compositions, and this is within the scope of the invention. The embodiments described herein are suitable for reacting spherical catalyst particles, preferably having a diameter from 3-8 mm.

End Caps

In some embodiments of the present invention, reactors 10 have end caps for providing various connections for the reaction cavities in the body 12 of the reactor 10. Various end cap designs are illustrated in FIG. 4A through FIG. 4F.

FIG. 4A through FIG. 4C show general features found in different embodiments end caps of the present invention. An end cap 400 of the present invention has a generally rectangular block shape and a first face 402 a second face 404 a third face 406 and a fourth face 408 as shown in FIG. 4A. The block has a surface defining two fluid cavities, each fluid cavity having an opening 410 412 on the first face 402. In FIG. 4 A through FIG. 4C, the openings 410 412 are shown generically as a circular opening. FIG. 4D through FIG. 4F show specific embodiments that use different opening designs depending on the desired configuration. The fluid cavities also have openings 422 424 on the fourth face 408. The fluid cavities are designed to allow fluid to flow into or out of the end cap 400 and into or out of the body 12 of the reactor 10. The first face also has threaded cavities 414 for receiving set screws. The cavities 414 extend through the end block 400 to the third face 406 and are used for attaching the end cap to another end cap or a sealing plate as shown in FIG. 5. The second face 404 has four cavities 416 also for receiving connection devices, such as set screws. The cavities 416 extend through the end cap 400 to the fourth face 408, and connection devices, such as set screws are used to attach the end cap 400 to an end 15 17 of the body 12. The cavities 416 are complementary to the cavities 48 located on the first 15 and second 17 ends of the body 12 shown in FIG. 2. In an assembled state, the fourth face 408 contacts the first end 15 or second end 17 of the body 12, and set screws are utilized in the cavities 416 of the end cap 400 and the cavities 48 of the block to establish a connection. The second face 404 also has a heat cartridge cavity 418 extending through the end block 400 which, when attached to a body 12, aligns with throughbore 30 of the body 12 for receiving a heater cartridge. Thermocouple cavities 422 also extend from the second face 404 to the fourth face 408 of the end cap 400 for receiving thermocouples. The thermocouple cavities 422 align with cavities 32 in the body 12 when assembled. The second face 404 also has a recess 420 that runs along the length of the face 404 for holding heater and thermocouple wires when they are utilized.

FIG. 4B is similar to FIG. 4A, but shows the connection cavities in phantom. The embodiment shown in FIG. 4B shows cavities for receiving set screws, the cavities having a wider opening at one face with a recessed sink for allowing the screws to extend through the end cap 400 and into complementing cavities. The screws are tightened through the wider openings as, when assembled, the faces 404 406 with the wider openings will not be in contact with any other surface.

FIG. 4C is similar to FIG. 4A, however the view of the end block 400 is reversed, showing the fourth face 408. FIG. 4C shows the fluid cavities in phantom as well as the heat cartridge cavity 418 in phantom. In general, the fluid cavities are designed with the cavities following a cylindrical shape from the fourth face 408 and turning into a conical shape that narrows as the cavity extends away from the fourth face 408. The fluid cavities have similar designs as they extend inward from the first face 402. Each fluid cavity has a ridge 426 428 that contacts the o-ring that sits in the groove created by the insert sleeve and/or the groove 50 in the body 12.

FIG. 4D through FIG. 4F show specific end cap designs for use in the present invention for achieving different reactor configurations.

FIG. 4D shows an inlet/outlet end cap 440 for use in connecting to a fluid source 34 and an analyzer 36. Fourth face 408 of end cap 440 is brought into mating connection with a first end 15 of the body 12 as described above. The inlet opening 442 on the first face 402 is adapted to connect to a fluid source, such as a vaporizer. In one embodiment, gas and liquid are mixed and vaporized in a vaporizer. The gas is then transported to the fluid cavity opening 442 via a heated transfer line. The opening 442 is adapted for sealed, pressurized, connection to the heated line. Various fluid connection means are known to those of skill in the art. The outlet opening 444 on the first face 402 is adapted for sealed, pressurized, connection to the to an analyzer, such as a gas chromatograph. In one embodiment, the opening is a threaded ferrule seal and is connected to a line that runs to a gas chromatograph. The end cap 440 is used to introduce a reactant fluid from a fluid source 34 into a first reaction cavity 18 of the reactor 10, and to discharge an effluent stream from a second reaction cavity 28. Depending on what other end caps are utilized on the opposite end of the body 12, the inlet 442 and outlet 444 may be part of a single reaction path, or may be parts of separate reactions. One difference between the end block 440 and the end block 400 described in FIG. 4B, is that the cavities 414 as shown have a reverse design, with the larger opening of the cavity being on the first face 402 and the smaller opening of the cavity 414 being on the third face 406, due to the fact that the first face 402 will not be contacting any other faces.

An inlet/outlet end cap 440 located at each end 15 17 of the body 12 provides a variation of the configuration described in FIG. 1D. Those of skill in the art will recognize, it is also within the scope of the invention to have an end cap with two inlets on one end 15 of the body 12 and an end cap with two outlets on the other end 17 of the body 12.

FIG. 4E shows a bypass end cap 460 for connecting two reaction cavities 18 28 located in a body 12. End cap 460 provides a connection channel 19, that, when sealed, allows fluid flow between reaction cavities 18 28. The fluid cavity designs are the same as those described generally. End cap 460 provides a recessed channel 19 connecting fluid cavity openings 462 464 on the first face 402. The first face 402 also has a recessed groove 466 surrounding the openings 462 464 and channel 19. The groove 466 is designed to hold an o-ring or other comparable sealing device. When the o-ring is placed in groove 466, a sealing plate, such as a plate 500 shown in FIG. 5 or the third face 406 of an end cap 400, is brought into contact with the first face 402 of end cap 460 to establish a bypass cavity 19, which allows fluid to flow from one reaction cavity 18 28 to the other reaction cavity in the reactor 10.

An inlet/outlet end cap 440 located at the first end 15 of the body 12 and a bypass end cap 460 located at the second end 17 of the body 12 provides the configuration described in FIG. 1C. A plurality of bodies 12 with this same design provide the configuration described in FIG. 1E.

FIG. 4F shows a flow through end cap 480 for connecting a reaction cavity from one body 12 with a reaction cavity in a second body 12. The fluid cavity openings 482 484 are surrounded by recessed grooves 486 488 designed to hold an o-ring or other comparable sealing device. The first face 402 of flow through end cap 480 is designed to contact a corresponding first face of a flow through end cap 480, resulting in the two reaction cavities in one body being separately connected to the reaction cavities in a second body.

Four Reaction Cavity Pathway Configuration

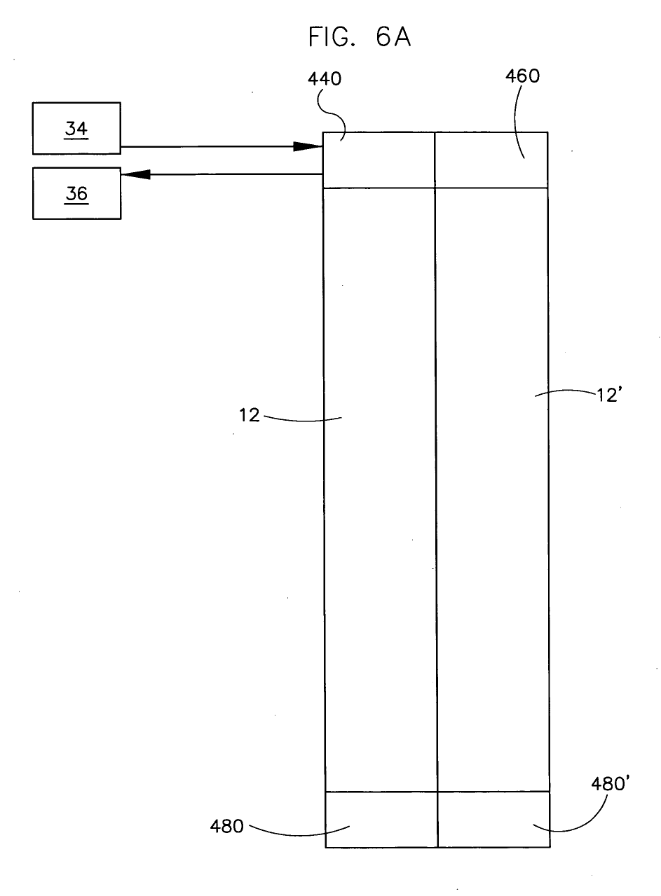

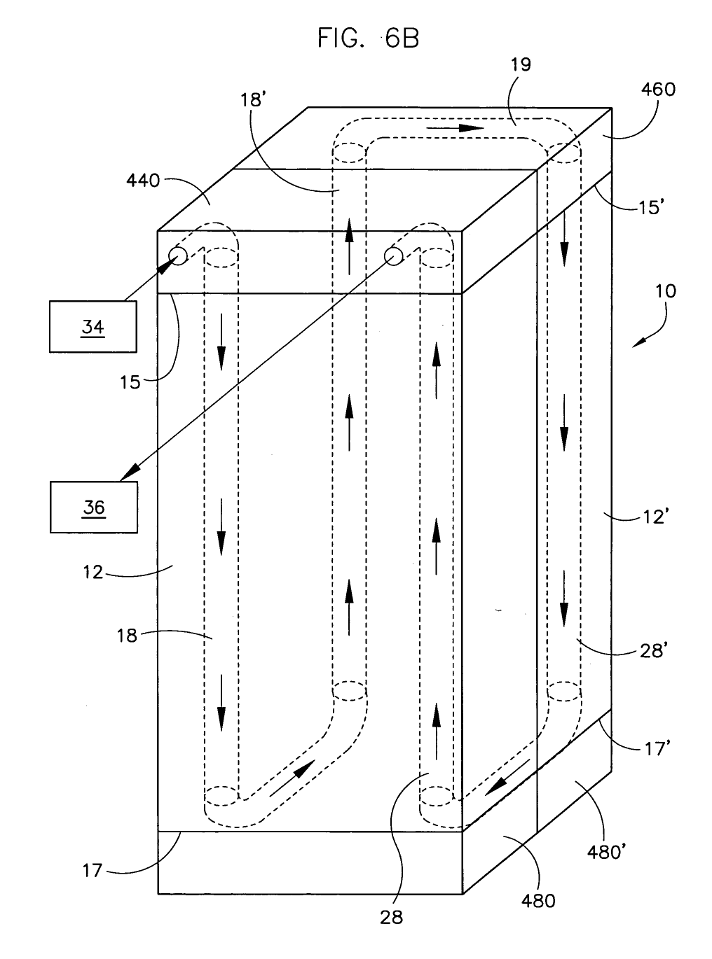

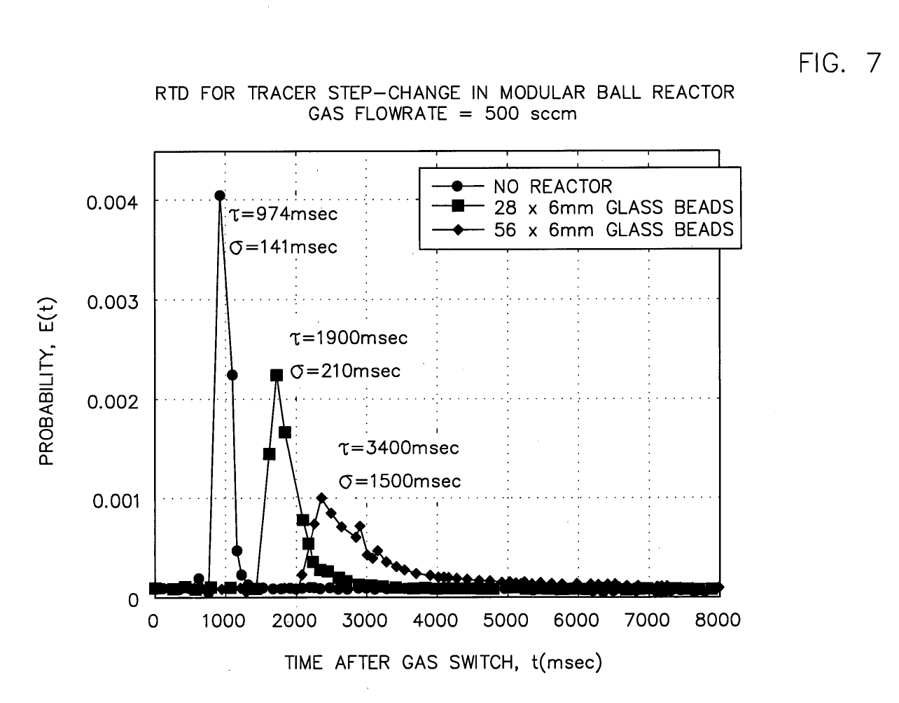

FIG. 6A and FIG. 6B show an embodiment of a reactor of the present invention configured for combining four reaction cavities into a single reaction pathway. The reactor 10 has a first body 12 having reaction cavities 18 28, and a second body 12′ having two reaction cavities 18′ 28′. The fourth face 408 of an inlet/outlet end block 440 is connected to the first end 15 of the first body 12 such that the third face 406 of the inlet/outlet end block 440 contacts and is connected to the first face 402 of a bypass end cap 460. The bypass end cap 460 which has the first face 402 contacting in connected to the third face 406 of the inlet/outlet end block 440, is situated such that it has its fourth face 408 in contact with and is connected to a first end 15′ of the second body 12′. The fourth face of a flow through end block 480 is in contact with and is connected to the second end 17 of body 12, such that the first face 402 of flow through end block 480 is in contact with and is connected to the first face 402 of flow through end block 480′. The flow through end block 480′, which has its first face 402 in contact with and is connected to the first face 402 of flow through end block 480 is situated such that it has its fourth face 408 in contact with and is connected to a second end 17′ of the second body 12′. The resulting reactor 10 has a reaction pathway, as indicated by arrows, that goes from the fluid source 34 through the inlet of the inlet/outlet end cap 440, through reaction cavity 18, into reaction cavity 18′ via flow through end caps 480 480′, through reaction cavity 18′ and into reaction cavity 28′ via bypass end cap 460, through reaction cavity 28′ and into reaction cavity 28 via flow through end caps 480 480′, through reaction cavity 28, and finally into the outlet of inlet/outlet end cap 440, where the effluent is discharged to an analyzer 36. This reactor provides longer residence time while maintaining small reactor size.

In operation, the chemical processing systems of the invention, can operate over various ranges of temperature, pressure, contact times and space velocities. For a chemical reaction system: the reactor temperature can generally range from about 0° C. to about 1000° C., and preferably from about 20° C. to about 500° C., and more preferably from about 100° C. to about 500° C.; the reactor pressure can range from about 1 bar to about 200 bar, and preferably from about 1 bar to about 10 bar; residence times can range from about 1 μsec to about 100 sec, preferably from about 1 μsec to about 10 seconds, and most preferably from about 0.2 seconds to about 5 seconds; and space velocities can range from about 1,000 hr⁻¹ to about 100,000 hr⁻¹, and preferably from about 1,000 hr⁻¹ to about 50,000 hr⁻¹, and more preferably from about 1,000 hr⁻¹ to about 10,000 hr⁻¹. For explosive reactants (e.g., hydrocarbons and oxygen), explosion limits should be observed.

The accompanying Figures and this description depict and describe embodiments of the reactor system and method of the present invention, and features and components thereof. Fastening, mounting, attaching or connecting the components of the present invention to form the apparatus or device as a whole, unless specifically described otherwise, are intended to encompass conventional fasteners such as machine screws, nut and bolt connectors, machine threaded connectors, snap rings, clamps such as screw clamps and the like, rivets, nuts and bolts, toggles, pins and the like. Components may also be connected by welding, friction fitting or deformation, if appropriate. Unless specifically otherwise disclosed or taught, materials for making components of the present invention are selected from appropriate materials such as metal, metallic alloys, fibers, plastics and the like, and appropriate manufacturing or production methods including casting, extruding, molding and machining may be used.

Any references herein to front and back, right and left, top and bottom, upper and lower and horizontal and vertical are intended for convenience of description only, not to limit the present invention or its components to any one positional or spatial orientation. Such terms are to be read and understood with their conventional meanings. In the Figures, elements common to the embodiments of the invention are commonly identified.

It is contemplated that various changes may be made without deviating from the spirit and scope of the present invention. Accordingly, it is intended that the scope of the present invention not be limited strictly to that of the above description of the present invention.

The following examples illustrate the principles and advantages of the invention.

### EXAMPLES

### Example 1

A modular ball reactor, such as that described above, having 7 mm diameter reaction cells, was used to confirm that the hydrodynamics of the reactor of the present invention perform comparably to a pilot or industrial scale fixed bed reactor. Calculated Peclet numbers for axial dispersion were used as a comparison. Helium gas was initially run through the reactor, and at time=0, the gas was switched to nitrogen. The time it took for the nitrogen to be detected at the outlet of the reactor corresponds to the residence time distribution (RTD) data listed in FIG. 7. the inserts were designed to hold 14 particles. Tests were run on a 28 particle system (using 6 mm glass beads to represent industrial form catalyst particles), using a configuration as shown in FIG. 1C, and on a 56 particle system, using a configuration as shown and described in FIG. 6B.

Results indicate a Pe=300 (±50) for 28×6 mm Glass Beads (One body) and a Pe=150 (±25) for 56×6 mm Glass Beads (Two bodies) (2× residence time)

This example shows that plug flow behavior can be obtained in the laboratory scale modular ball reactor using commercial form catalyst pellets.

Residence time distribution in this example shows that the hydrodynamics perform comparably to a pilot or industrial scale fixed bed reactor.

### Example 2

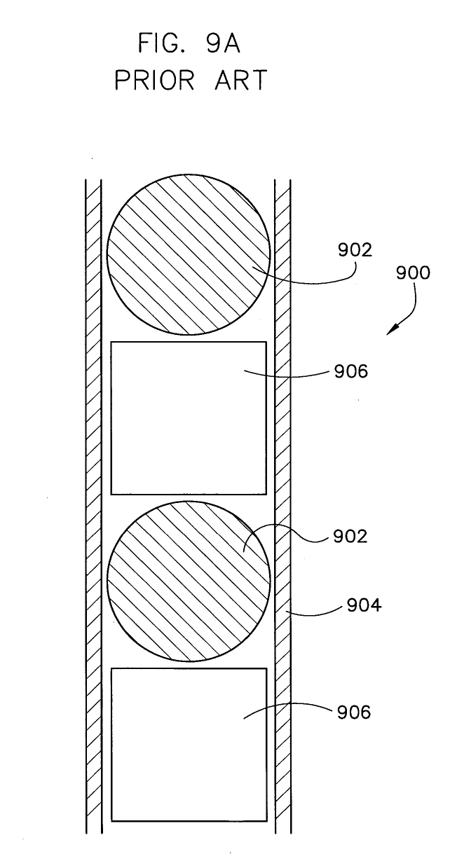

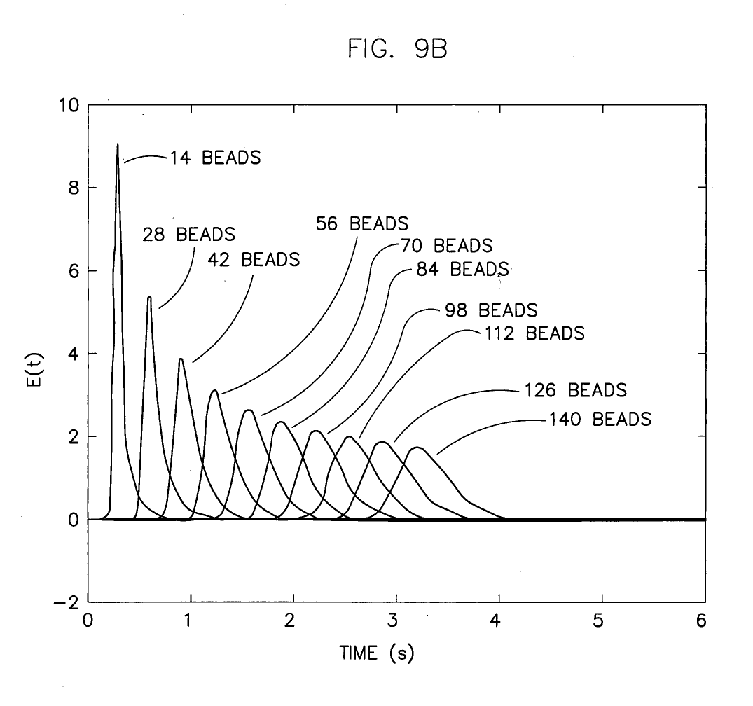

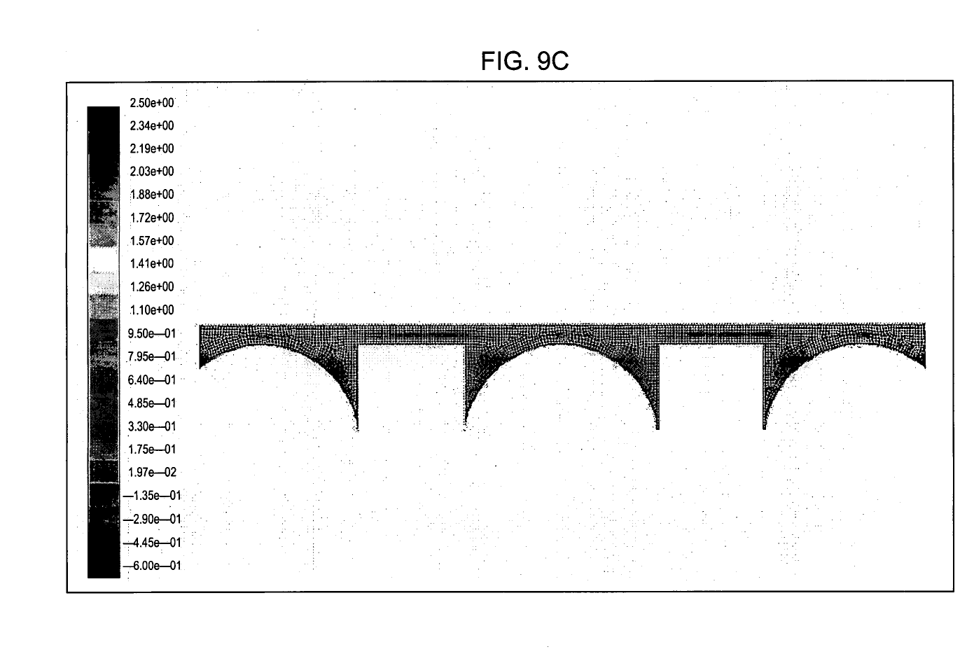

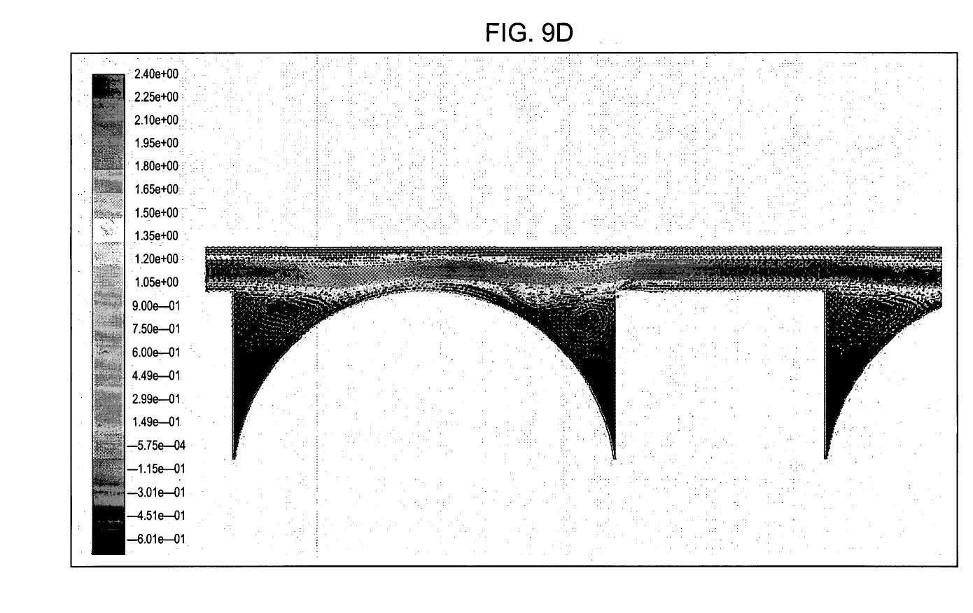

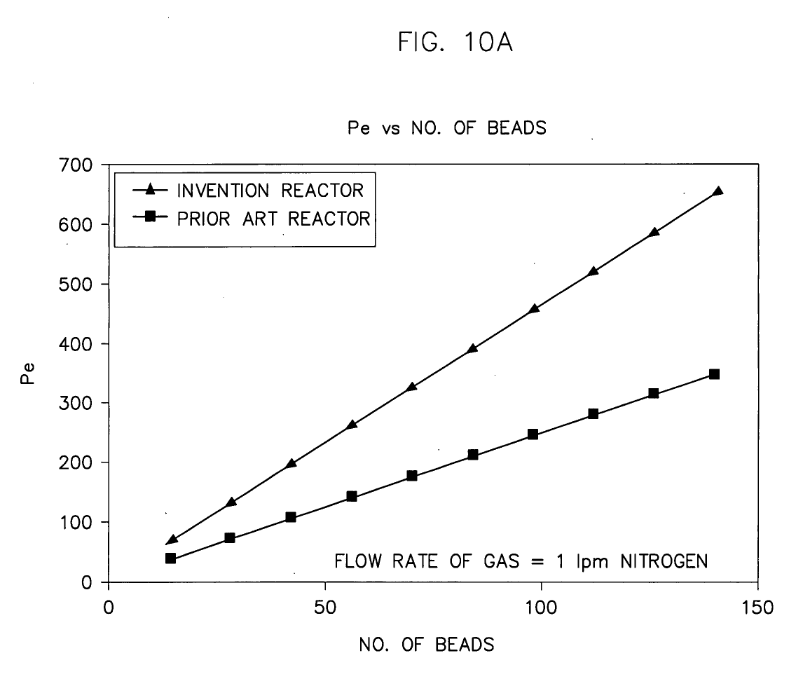

In this example, experiments were conducted to determine and compare the residence time distribution (RTD) and velocity profiles in the prior art reactor described by Temkin as shown in FIG. 9A and a modular ball reactor as described above. The reactor 900 shown in FIG. 9A comprises a plurality of beads or catalyst particles 902 located in a tube 904 and spaced apart by inert cylindrical spacers 906.

The experiments were performed using FLUENT computational fluid dynamics software. Both 2D and 3D simulations were performed on both designs and after validating the 2D results with the 3D results, the 2D models were used to reduce computation time.

Nitrogen was used as the process gas to model fluid flow and was run at 1 liter/minute. The RTD experiments were performed as step experiments. While the system was at steady state, flow was switched from a tracer nitrogen species (having exactly the same physical properties as nitrogen) to nitrogen and the concentration of nitrogen in the gas was measured at the inlet and outlet of the reactor.

Experiments were performed using 14 6 mm diameter beads in each reactor, the beads representing catalyst particles, and the results were convoluted to determine the RTD for an increasing number of beads.

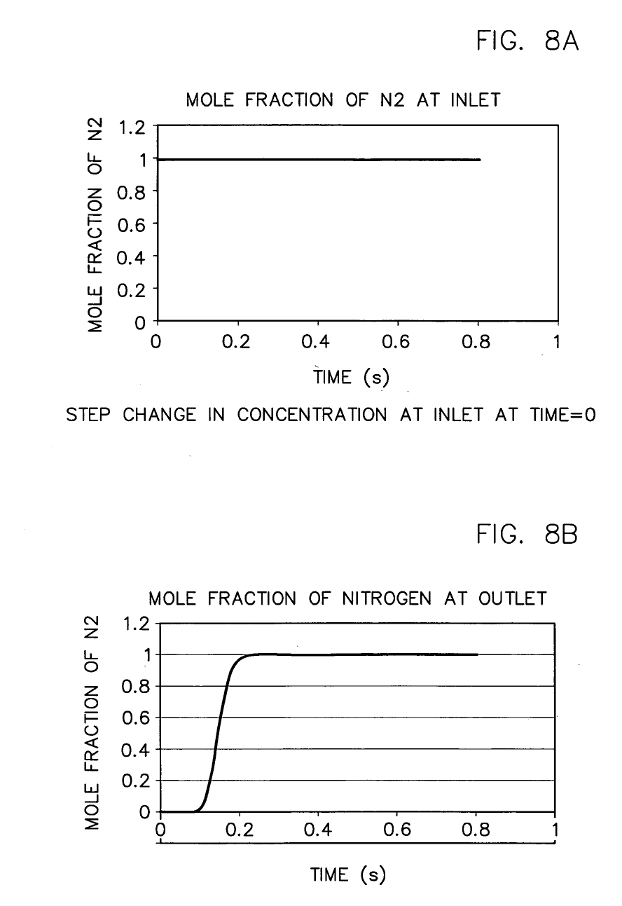

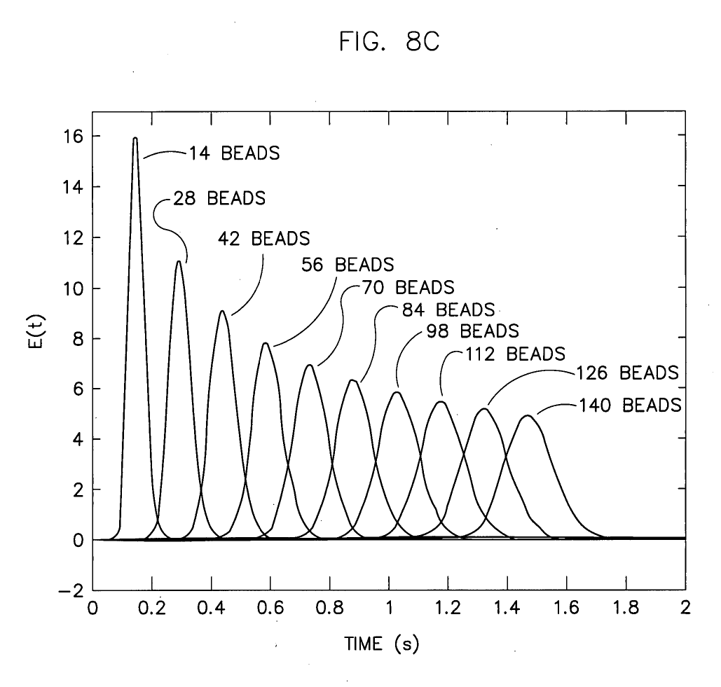

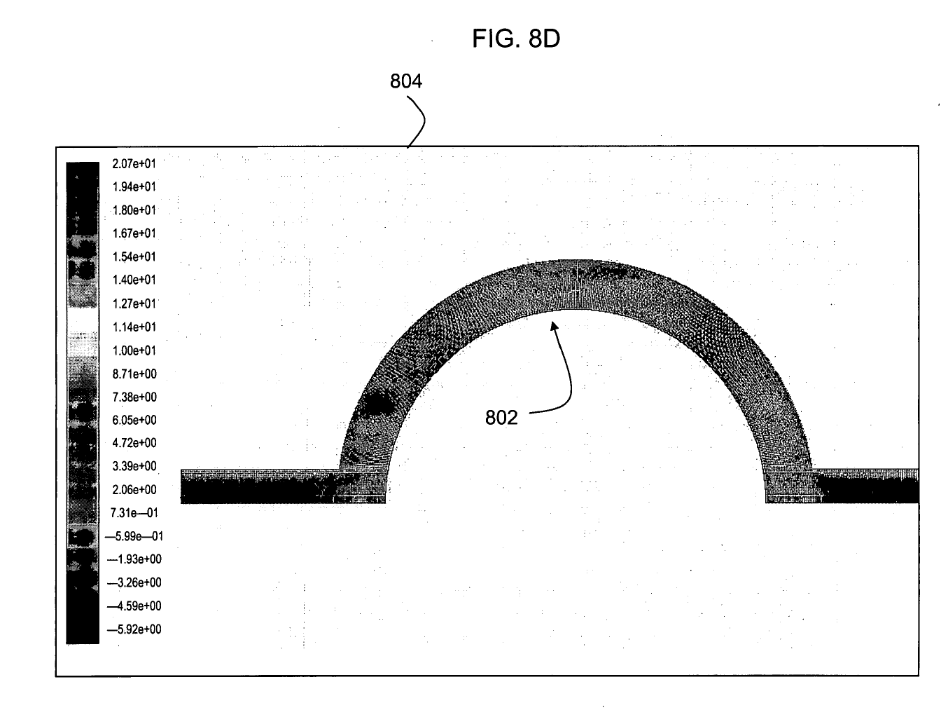

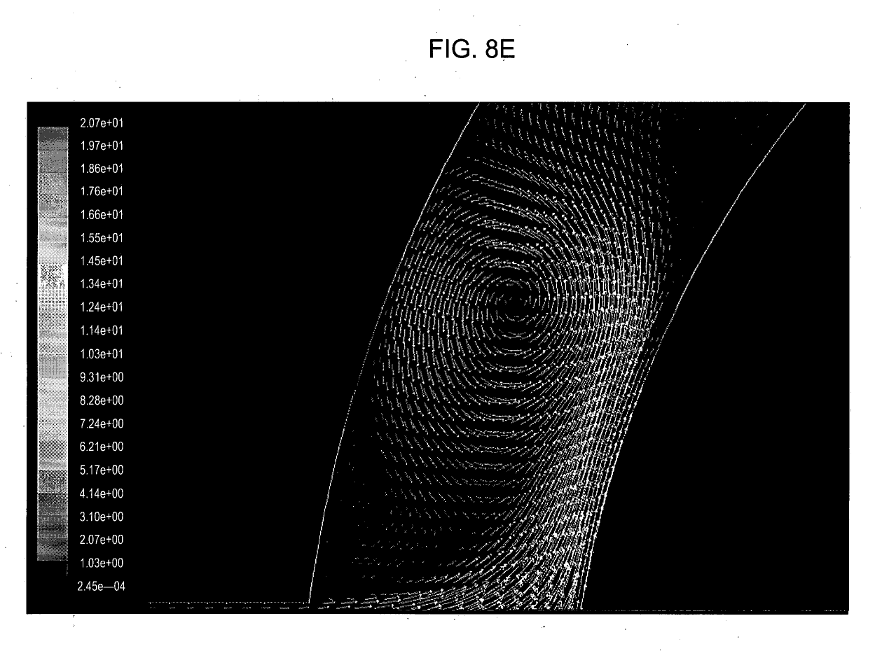

The mole fractions of nitrogen at the inlet and outlet of the reactor of the invention as a function of time is shown in FIG. 8A and FIG. 8B, respectively. T=0 indicates the time at which the gas was changed from the tracer nitrogen species to nitrogen.

The results were convoluted to determine the RTD for the reactor of the invention using an increasing number of beads (representing catalyst particles). The results are shown in FIG. 8C, which indicates the calculated RTD for the reactor having up to 140 beads, or catalyst particles.

The same procedures were then run on the reactor shown in FIG. 9A, using 14 6 mm beads and 14 cubic spacers.

The results were convoluted to determine the RTD for the prior art reactor using an increasing number of beads (representing catalyst particles). The results are shown in FIG. 9B, which indicates the calculated RTD for the reactor having up to 140 beads, or catalyst particles.

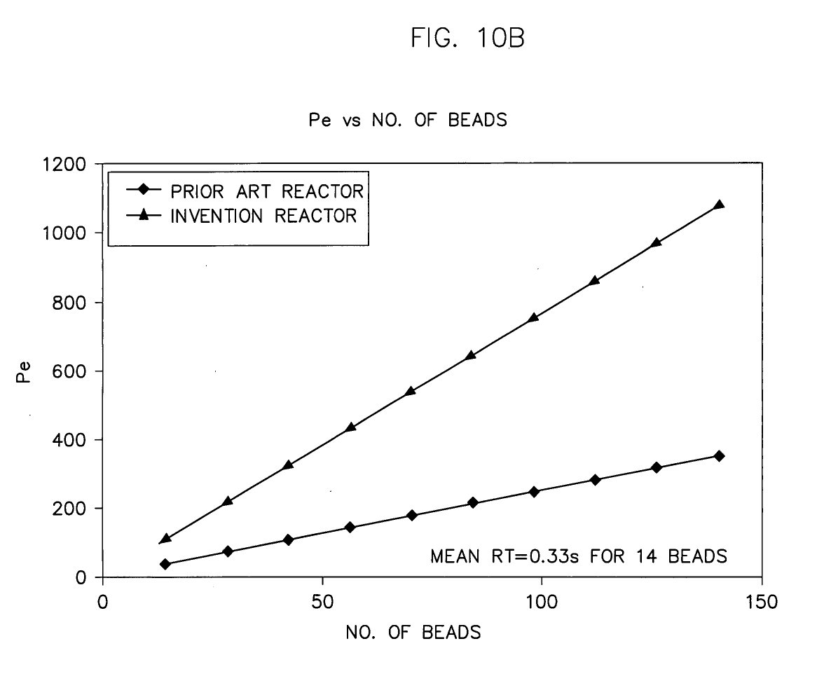

Peclet numbers were then calculated and compared for an increasing number of beads used in the reactor of the invention as compared to the prior art reactor. FIG. 10A shows the comparison of Peclet number as a function of number of beads under a similar flow rate of 1 liter/minute. FIG. 10B shows the comparison of calculated Peclet number as a function of number of beads using a similar residence time for each reactor.

With this data and the reactor designs, velocity profiles were simulated using Fluent computational fluid dynamics software. FIG. 8D shows the simulated axial velocity profile between a single bead 802 and the reactor wall 804 in the reactor of the invention, and FIG. 8E shows a blown up view of the profile shown in FIG. 8D. FIG. 9C shows the simulated axial velocity profile between a bead 902 and the reactor wall 904 in the prior art reactor, and FIG. 9D shows a blown up view of the profile shown in FIG. 9C.

The reactor of the present invention showed a nearly uniform velocity profile over the catalyst bead under reaction conditions. No dead zones were present, whereas the velocity profiles for the reactor described by the prior art showed the presence of dead zones and non-uniform velocity distribution over the surface of the catalyst bead under similar reaction conditions. The reactor of the present invention showed a better residence time distribution with higher Peclet numbers indicating better plug flow behavior in comparison to the reactor described by Temkin. Thus, the reactor design of the present invention is superior to the reactor described by Temkin in terms of velocity distribution, plug flow behavior and external mass transport.

In light of the detailed description of the invention and the example presented above, it can be appreciated that the several objects of the invention are achieved.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

We claim:

1. A method for evaluating catalysts, the method comprising:
flowing a reactant fluid through a reactor comprising a surface defining a reaction cavity, the reaction cavity having a volume less than 1 liter, wherein the reactant fluid contacts a plurality of solid catalyst particles located in the reaction cavity under reaction conditions such that a flow characteristic of the reactant fluid through the reaction cavity of the reactor is a Peclet number for axial dispersion greater than 150.

2. The method of claim 1, wherein the solid catalyst particles all have dimensions suitable for industrial scale processes.

3. The method of claim 1, wherein the reactant fluid has a substantially uniform velocity over substantially the entire surface of each catalyst particle during the flowing step.

4. The method of claim 1, wherein the reaction conditions comprise a reactor temperature greater than 100° C.

5. The method of claim 1, wherein the reaction conditions comprise a reactor pressure greater than about 10 bar.

6. The method of claim 1, wherein the reaction conditions comprise a space velocity in the range from about 1,000 hr⁻¹ to about 100,000 hr⁻¹.

7. The method of claim 1, wherein the catalysts are spherical and have a diameter between about 3 mm and about 8 mm.

8. The method of claim 1, wherein the reaction cavity has a volume less than 100 mL.

9. The method of claim 1, wherein the flowing step comprises flowing the reactant fluid out of the reactor as an effluent after contacting the plurality of solid catalyst particles, the method further comprising analyzing the effluent with a detector.

10. A heterogeneous catalysis process comprising
contacting a gas stream in a reactor comprising a surface defining a reaction cavity, the reaction cavity having a volume less than 1 liter, with a plurality of solid catalyst particles located in the reaction cavity, the catalyst particles having dimensions suitable for industrial scale processes, under reaction conditions such that the gas stream has a substantially uniform velocity at all points over a surface of the plurality of solid catalyst particles.

11. The process of claim 10, wherein a flow characteristic of the reactant fluid through the reaction cavity of the reactor is a Peclet number for axial dispersion greater than 100.

12. A method for evaluating catalysts, the method comprising:
providing a reactor comprising
a surface defining an internal cavity, and
an insert located in the internal cavity, the insert comprising
a fluid inlet for receiving a fluid from a fluid source,
a fluid outlet for discharging the fluid as an effluent,
a surface defining a plurality of reaction cells connected via conduits, and
a plurality of industrial form solid catalyst particles located in the reaction cells, wherein each reaction cell is adapted to hold a single catalyst particle, and
flowing a reactant fluid through the reaction cells, wherein the reactant fluid contacts the plurality of solid catalyst particles located in the reaction cells under reaction conditions.

13. The method of claim 12, wherein a flow characteristic of the reactant fluid through the reaction cells is a Peclet number for axial dispersion greater than 100.

14. The method of claim 12, wherein the plurality of industrial form solid catalyst particles are spherical and have a diameter between about 3 mm and about 8 mm.

15. A system for contacting solid catalyst particles with a fluid, the system comprising
a reactor adapted to hold a plurality of solid catalyst particles, the reactor comprising
a surface defining an internal cavity, and
an insert located in the internal cavity, the insert comprising
a fluid inlet for receiving a fluid,
a fluid outlet for discharging the fluid, and
a surface defining a reaction cavity running from the inlet to the outlet, the reaction cavity comprising a plurality of reaction cells connected via conduits, wherein each reaction cell is adapted to hold a single catalyst particle.

16. The system of claim 15, further comprising a fluid source in fluid communication with the fluid inlet of the reactor for providing fluid flow through the reaction cavity.

17. The system of claim 15, further comprising a detector in fluid communication with the outlet of the reactor for analyzing reactor effluent.

18. The system of claim 15, wherein the catalysts are spherical and have a diameter between about 3 mm and about 8 mm.

19. The system of claim 15, wherein the plurality of reaction cells connected via conduits has a total volume less than 1 liter.

20. The system of claim 15, wherein the surface defining an internal cavity is a first surface defining a first internal cavity, the insert is a first inert insert, the inlet is a first inlet, the outlet is a first outlet, and the surface defining the plurality of reaction cells connected via conduits is a first surface defining a first plurality of reaction cells connected via conduits, the reactor further comprising
a second surface defining a second internal cavity, and
a second insert located in the second internal cavity, the second insert comprising
a second fluid inlet for receiving a fluid,
a second fluid outlet for discharging the fluid, and
a second surface defining a second reaction cavity running from the second inlet to the second outlet, the second reaction cavity comprising a second plurality of reaction cells connected via conduits, wherein each reaction cell is adapted to hold a single catalyst particle.

21. The system of claim 15, further comprising a plurality of spacers located in the plurality of reaction cells for holding the catalyst particles.

22. The system of claim 15, wherein the reactor is adapted to hold no more than 100 catalyst particles.

23. The system of claim 15, wherein the catalyst particles are industrial form catalysts.

24. A reactor system comprising
a vessel comprising:
an inlet for receiving a fluid
an outlet for discharging the fluid as an effluent,
a surface defining a reaction cavity having a volume less than 1 liter, and
a plurality of industrial form catalyst particles located in the reaction cavity, wherein the reaction cavity is adapted to provide a substantially uniform velocity of the fluid over the surface of each of the catalyst particles under reaction conditions.

25. The reactor system of claim 24, further comprising a fluid source in fluid communication with the inlet of the vessel for providing fluid flow through the flow path of the vessel.

26. The reactor system of claim 24, further comprising a detector in fluid communication with the outlet of the vessel for analyzing vessel effluent.

27. The reactor system of claim 24, wherein the catalyst particles are spherical and have a diameter between about 3 mm and about 8 mm.

28. The reactor system of claim 24, wherein the vessel is a first vessel, the inlet is a first inlet, the outlet is a first outlet, the surface defining a reaction cavity is a first surface defining a first reaction cavity, and the plurality of industrial form catalyst particles is a first plurality of catalyst particles, the reactor further comprising
a second vessel comprising:
a second inlet for receiving a fluid
a second outlet for discharging the fluid as an effluent,
a second surface defining a second reaction cavity having a volume less than 1 liter, and
a second plurality of industrial form catalyst particles located in the second reaction cavity, wherein the second reaction cavity is adapted to provide a substantially uniform velocity of the fluid over the surface of each of the catalyst particles under reaction conditions.

29. The reactor system of claim 24, wherein the plurality of industrial form catalyst particles comprises less than 100 catalyst particles.

30. A reactor system for evaluating catalysts, the reactor comprising
a vessel comprising
an inlet for receiving a fluid,
an outlet for discharging the fluid,
a surface defining a reaction cavity, and
a plurality of solid catalyst particles all having approximately the same geometry and dimensions located in the reaction cavity, wherein the reaction cavity comprises:
a cross sectional area no greater than twice a cross-sectional area of a single catalyst particle of the plurality of catalysts, and
a length adapted to accommodate the plurality of catalyst particles, wherein the reaction cavity is adapted to provide a substantially uniform velocity of the fluid over substantially the entire surface of the plurality of catalyst particles under reaction conditions.

31. The reactor system of claim 30, wherein the catalysts are spherical and have a diameter between about 3 mm and about 8 mm.

32. The reactor system of claim 30, wherein the reaction cavity has a volume less than 1 liter.

33. The reactor system of claim 30, wherein the vessel is a first vessel, the inlet is a first inlet, the outlet is a first, the surface defining the reaction cavity is a first surface defining a first reaction cavity and the plurality of solid catalyst particles is a first plurality of solid catalyst particles, the reactor further comprising
a second vessel comprising
a second inlet for receiving a fluid,
a second outlet for discharging the fluid,
a second surface defining a second reaction cavity, and
a second plurality of solid catalyst particles all having approximately the same geometry and dimensions located in the second reaction cavity, wherein the second reaction cavity comprises:
a cross sectional area no greater than twice a cross-sectional area of a single catalyst particle of the second plurality of catalysts, and a length adapted to accommodate the second plurality of catalyst particles, wherein the second reaction cavity is adapted to provide a substantially uniform velocity of the fluid over substantially the entire surface of the second plurality of catalyst particles under reaction conditions.

34. The reactor system of claim 30, wherein the plurality of solid catalyst particles comprises less than 100 catalyst particles.

35. A parallel flow reactor system for evaluating a plurality of industrial form catalyst particles, the reactor comprising
a plurality of surfaces defining a plurality of reaction cavities, each of the plurality of reaction cavities comprising an inlet for receiving a reactant-containing stream and an outlet for discharging a product-containing stream, wherein each reaction cavity comprises a plurality of industrial form solid catalysts,
the reactor system being adapted such that each reactant-containing stream can be fed through the plurality of reaction cavities simultaneously to contact the catalyst particles under reaction conditions and wherein each reaction cavity is adapted to provide a substantially uniform velocity of the reactant-containing stream over substantially the entire surface of the plurality of catalyst particles under reaction conditions.
